(19) United States
(12) Patent Application Publication
(10) Pub. No.: US 2018/0147561 A1
(43) Pub. Date: May 31, 2018

(54) METHOD FOR PRODUCING CATALYSTS CONTAINING CHROME, FOR THE OXIDATIVE DEHYDROGENATION OF N-BUTENES TO FORM BUTADIENE WHILE AVOIDING CR(VI) INTERMEDIATES

(71) Applicant: BASF SE, Ludwigshafen am Rhein (DE)

(72) Inventors: Jan UNGELENK, Neuhofen (DE); Ulrich HAMMON, Mannheim (DE); Kazuhiko AMAKAWA, Mannheim (DE); Philipp GRÜNE, Mannheim (DE); Christian WALSDORFF, Ludwigshafen (DE)

(21) Appl. No.: 15/571,649

(22) PCT Filed: May 4, 2016
(86) PCT No.: PCT/EP2016/059963

§ 371 Date: Nov. 3, 2017

(30) Foreign Application Priority Data
May 6, 2015 (EP) 15166567.6

(51) Int. Cl. B01J 23/28; B01J 23/887; B01J 37/08; B01J 37/00; C07C 5/48; B01J 37/04; B01J 23/31; B01J 35/02; B01J 37/02

(52) U.S. Cl. CPC B01J 23/28; B01J 23/8878; B01J 37/08; B01J 37/0036; C07C 5/48; B01J 37/0221; B01J 23/31; B01J 35/023; B01J 37/0219; B01J 2523/00; C07C 2523/887; B01J 37/04

(57) ABSTRACT

Process for producing a multimetal oxide catalyst comprising molybdenum, chromium and at least one further metal by mixing of a pulverulent multimetal oxide comprising molybdenum and at least one further metal but no chromium with pulverulent chromium(III) oxide and thermal treatment of the resulting pulverulent mixture in the presence of oxygen at a temperature in the range from 350° C. to 650° C.

The invention relates to a catalyst, in particular a mixed oxide catalyst, for the oxidative dehydrogenation of n-butenes to butadiene, the production thereof, the use thereof and also a process for the oxidative dehydrogenation of n-butenes to 1,3-butadiene (butadiene).

Butadiene is an important basic chemical and is used, for example, for producing synthetic rubbers (butadiene homopolymers, styrene-butadiene rubber or nitrile rubber) or for producing thermoplastic terpolymers (acrylonitrile-butadiene-styrene copolymers). Butadiene is also converted into sulfolane, chloroprene and 1,4-hexamethylenediamine (via 1,4-dichlorobutene and adiponitrile). Furthermore, dimerization of butadiene can produce vinylcyclohexene which can be dehydrogenated to styrene.

Butadiene can be prepared by thermal cracking (steam cracking) of saturated hydrocarbons, usually starting out from naphtha as raw material. The steam cracking of naphtha gives a hydrocarbon mixture composed of methane, ethane, ethene, acetylene, propane, propene, propyne, allene, butanes, butenes, butadiene, butynes, methylallene, C₅-hydrocarbons and higher hydrocarbons.

Butadiene can also be obtained by oxidative dehydrogenation of n-butenes (1-butene and/or 2-butene). As starting gas mixture for the oxidative dehydrogenation of n-butenes to butadiene, it is possible to use any mixture comprising n-butenes. For example, it is possible to use a fraction which comprises n-butenes (1-butene and/or 2-butene) as main constituent and has been obtained from the C₄ fraction from a naphtha cracker by removal of butadiene and isobutene. Furthermore, gas mixtures which comprise 1-butene, cis-2-butene, trans-2-butene or mixtures thereof and have been obtained by dimerization of ethylene can also be used as starting gas. Gas mixtures which comprise n-butenes and have been obtained by fluid catalytic cracking (FCC) can also be used as starting gas.

Gas mixtures which comprise n-butenes and are used as starting gas in the oxidative dehydrogenation of n-butenes to butadiene can also be produced by nonoxidative dehydrogenation of n-butane-comprising gas mixtures.

WO 2005/063658 discloses a process for preparing butadiene from n-butane, which comprises the steps

A) provision of an n-butane-comprising feed gas stream a;

B) introduction of the n-butane-comprising feed gas stream a into at least one first dehydrogenation zone and nonoxidative catalytic dehydrogenation of n-butane to give a product gas stream b comprising n-butane, 1-butene, 2-butene, butadiene, hydrogen, low-boiling secondary constituents and possibly water vapor;

C) introduction of the product gas stream b from the nonoxidative catalytic dehydrogenation and an oxygen-comprising gas into at least one second dehydrogenation zone and oxidative dehydrogenation of 1-butene and 2-butene to give a product gas stream c which comprises n-butane, 2-butene, butadiene, hydrogen, low-boiling secondary constituents and water vapor and has a higher butadiene content than the product gas stream b;

D) removal of hydrogen, the low-boiling secondary constituents and water vapor to give a C₄ product gas stream d consisting essentially of n-butane, 2-butene and butadiene;

E) separation of the C₄ product gas stream d into a recycle stream e1 consisting essentially of n-butane and 2-butene and a stream e2 consisting essentially of butadiene by extractive distillation and recirculation of the stream e1 into the first dehydrogenation zone.

WO 2009/124945 discloses a coated catalyst for the oxidative dehydrogenation of 1-butene and/or 2-butene to butadiene, which is obtained from a catalyst precursor comprising

(a) a support body,

(b) a coating comprising (i) a catalytically active multimetal oxide comprising molybdenum and at least one further metal and having the general formula

Mo₁₂BiₐCr_{b}X¹_{c}Fe_{d}X²ₑX³_{f}O_{y}

where
X¹=Co and/or Ni,
X²=Si and/or Al,
X³=Li, Na, K, Cs and/or Rb,
0.2≤a≤1,
0≤b≤2,
2≤C≤10,
0.5≤d≤10,
0≤e≤10,
0≤f≤0.5 and
y=a number determined by the valence and abundance of the elements other than oxygen in order to maintain electrical neutrality,
and (ii) at least one pore former.

As support bodies for the coated catalysts, use is made of steatite balls having a diameter of from 2 to 3 mm.

WO 2014/086831 discloses a catalyst which is obtainable from a catalyst precursor comprising a catalytically active multimetal oxide comprising molybdenum and at least one further metal and having the general formula (I)

Mo₁₂BiₐFe_{b}Co_{c}Ni_{d}CrₑX_{1f}X_{2g}Oₓ (I),

where the variables having the following meanings:

X¹=W, Sn, Mn, La, Ce, Ge, Ti, Zr, Hf, Nb, P, Si, Sb, Al, Cd and/or Mg;

X²=Li, Na, K, Cs and/or Rb,

a=0.1 to 7, preferably from 0.3 to 1.5;

b=0 to 5, preferably from 2 to 4;

c=0 to 10, preferably from 3 to 10;

d=0 to 10;

e=0 to 5, preferably from 0.1 to 2;

f=0 to 24, preferably from 0.1 to 2;

g=0 to 2, preferably from 0.01 to 1; and

x=a number determined by the valence and abundance of the elements other than oxygen in (I),

wherein the catalyst has the shape of a hollow cylinder, where the internal diameter is from 0.2 to 0.8 times the external diameter and the length is from 0.5 to 2.5 times the external diameter, and the catalyst precursor does not comprise any pore former.

WO 2010/137595 discloses a multimetal oxide catalyst for the oxidative dehydrogenation of alkenes to dienes, which comprises at least molybdenum, bismuth and cobalt and has the general formula

MoₐBi_{b}Co_{c}Ni_{d}FeₑX_{f}Y_{g}ZₕSiᵢOⱼ

In this formula, X is at least one element from the group consisting of magnesium (Mg), calcium (Ca), zinc (Zn), cerium (Ce) and samarium (Sm). Y is at least one element from the group consisting of sodium (Na), potassium (K), rubidium (Rb), cesium (Cs) and thallium (TI). Z is at least one element from the group consisting of boron (B), phosphorus (P), arsenic (As) and tungsten (W). a-j are the atom fraction of the respective element, where a=12, b=0.5-7, c=0-10, d=0-10, (where c+d=1-10), e=0.05-3, f=0-2, g=0.04-2, h=0-3 and I=5-48. No more detailed information is given on the geometry of the shaped catalyst bodies. In the examples, a catalyst having the composition Mo₁₂Bi₅Co_{2.5}Ni_{2.5}Pe_{0.4}Na_{0.35}B_{0.2}K_{0.08}Si₂₄ is used in the form of pellets having a diameter of 5 mm and a height of 4 mm in the oxidative dehydrogenation of n-butenes to butadiene.

The catalytically active multimetal oxide composition can comprise chromium(III) oxide. As starting materials, it is possible to use not only the oxides but, in particular, also halides, nitrates, formates, oxalates, acetates, carbonates and/or hydroxides. The thermal decomposition of chromium(III) compounds into chromium(III) oxide occurs independently of the presence or absence of oxygen, mainly in the range 70-430° C. via a plurality of chromium(VI)-comprising intermediates (see, for example, J. Therm. Anal. Cal., 72, 2003, 135 and Env. Sci. Tech. 47, 2013, 5858). The presence of chromium(VI) oxide is not necessary for the catalytic oxydehydrogenation of alkenes to dienes, especially of butenes to butadiene. Owing to the toxicity and environmentally damaging nature of Cr(VI) oxide, the active composition should therefore be largely free of chromium(VI) oxide. Owing to their toxicity, chromium(VI) compounds should also not be formed as intermediates during production of the catalysts.

It is an object of the invention to provide a process for producing chromium(VI)-free, chromium-comprising catalysts for the oxydehydrogenation of butenes to butadiene, in which essentially no chromium(VI) compounds are formed as intermediate during the production of the catalysts.

This object is achieved by a process for producing a multimetal oxide catalyst comprising molybdenum, chromium and at least one further metal by mixing of a pulverulent multimetal oxide comprising molybdenum and at least one further metal but no chromium with pulverulent chromium(III) oxide and thermal treatment of the resulting pulverulent mixture in the presence of oxygen at a temperature in the range from 350° C. to 650° C., preferably from 400° C. to 600° C.

The multimetal oxide is considered to comprise no chromium when its chromium content is less than 50 ppm, based on the mass of the multimetal oxide.

According to the invention, the active compositions of the corresponding catalysts are firstly produced in a form completely free of chromium. This chromium-free multimetal oxide active composition is subsequently mixed with an amount of chromium(III) oxide Cr₂O₃ corresponding to the target stoichiometry and the mixture is subjected to a solid-state reaction. Solid-state reactions usually require relatively high temperatures of, for example, from 1000° C. to 1500° C. and long reaction times of >1d. It has surprisingly been found that comparatively low temperatures of only about 500° C. (melting point of Cr₂O₃=2435° C.) and short reaction times (<1d) are sufficient to bring about the solid-state reaction without hexavalent intermediates of chromium being formed. Nonoxidic Cr(III) compounds on the other hand go through hexavalent intermediates in the solid-state reaction.

The temperature at which the thermal treatment is carried out is preferably from 400 to 600° C., in particular from 450 to 550° C. The treatment time is generally from 2 to 24 hours, preferably from 3 to 8 hours. The thermal treatment is carried out in the presence of an oxygen-comprising gas, preferably in the presence of air.

In general, the production of the pulverulent multimetal oxide which comprises molybdenum and at least one further metal but no chromium comprises the steps (i) to (iv):

(i) production of a multimetal oxide precursor composition comprising molybdenum and at least one further metal but no chromium,

(ii) shaping of shaped bodies from the multimetal oxide precursor composition,

(iii) calcination of the shaped bodies,

(iv) milling of the shaped bodies to give a pulverulent multimetal oxide,

The catalyst of the invention can be an all-active catalyst or a coated catalyst. It is preferably a coated catalyst.

In a preferred embodiment, the process of the invention comprises the steps (v) to (viii):

(v) mixing of the pulverulent multimetal oxide comprising molybdenum and at least one further metal but no chromium with pulverulent chromium(III) oxide,

(vi) thermal treatment of the pulverulent mixture in the presence of oxygen at a temperature of from 350 to 650° C., in particular from 450 to 550° C. to give a pulverulent multimetal oxide catalyst comprising molybdenum, chromium and at least one further metal,

(vii) coating of a support body with the pulverulent multimetal oxide catalyst, (viii) thermal treatment of the coated support body.

The multimetal oxide comprising molybdenum and at least one further metal but no chromium generally has the general formula (I)

Mo₁₂BiₐFe_{b}Co_{c}Ni_{d}X¹_{f}X²_{g}Oₓ (I)

where the variables have the following meanings:
X¹=W, Sn, Mn, La, Ce, Ge, Ti, Zr, Hf, Nb, P, Si, Sb, Al, Cd and/or Mg;
X²=Li, Na, K, Cs and/or Rb,
a=0.1 to 7, preferably from 0.3 to 1.5;
b=0 to 10, preferably from 2 to 4;
c=0 to 10, preferably from 3 to 10;
d=0 to 10, preferably 0;
f=0 to 50, preferably from 0.1 to 10;
g=0 to 2, preferably from 0.01 to 1; and
x=a number determined by the valence and abundance of the elements other than oxygen in (I),

and the chromium(VI)-free multimetal oxide catalyst of the invention comprising molybdenum, chromium and at least one further metal has the general formula (II)

Mo₁₂BiₐFe_{b}Co_{c}Ni_{d}CrₑX¹_{f}X²_{g}O_{y} (II)

where the variables have the following meanings:
X¹=W, Sn, Mn, La, Ce, Ge, Ti, Zr, Hf, Nb, P, Si, Sb, Al, Cd and/or Mg;
X²=Li, Na, K, Cs and/or Rb,
a=0.1 to 7, preferably from 0.3 to 1.5;
b=0 to 10, preferably from 2 to 4;
c=0 to 10, preferably from 3 to 10;
d=0 to 10, preferably 0;
e=>0 to 5, preferably from 0.1 to 2;
f=0 to 50, preferably from 0.1 to 10;
g=0 to 2, preferably from 0.01 to 1; and
y=a number determined by the valence and abundance of the elements other than oxygen in (II).

The invention also provides the chromium(VI)-free multimetal oxide catalysts comprising molybdenum, chromium and at least one further metal themselves. The catalyst is considered to be chromium(VI)-free when its content of chromium(VI) is less than 200 ppm, based on the composition.

The content of chromium(VI) is determined by photometry. The determination method follows DIN 53780 DE.

For this purpose, from about 0.4 to 0.6 g of the sample are weighed to within 0.1 mg into a 100 ml volumetric flask and made up to the mark with water. After stirring for 4 hours on a magnetic stirrer, the sample is filtered through a fluted filter (“water withdrawal”).

An aliquot of the solution (depending on content) is acidified with phosphoric acid and admixed with an excess of 1,5-diphenylcarbazide (about 1 ml) based on Cr⁶⁺. The blank determination is carried out analogously only without a sample of the catalyst to be tested. After standing for 15 minutes, these solutions are measured photometrically and the content is determined taking into account the blank (parameters: curvette: 50 mm; wavelength: 540 nm; instrument: photometer, e.g. UV mini 1240, from Shimadzu).

The catalyst preferably has the shape of a hollow cylinder, with the internal diameter being from 0.2 to 0.8 times the external diameter and the length being from 0.5 to 2.5 times the external diameter. Catalysts in the form of a hollow cylinder have a particularly low pressure drop, so that the oxidative dehydrogenation can be carried out at an overall lower pressure. It has been found that the formation of carbonaceous material precursors is pressure-dependent. Thus, the formation of particular carbonaceous material precursors such as styrene, anthraquinone and fluorenone increases overproportionally at pressures at the reactor inlet above 1.3 bar absolute.

The catalyst according to the invention can be an all-active catalyst or a coated catalyst. If it is a coated catalyst, it has a support body (a) and a coating (b) comprising the catalytically active multimetal oxide of the general formula (II) comprising molybdenum and at least one further metal.

Preferred catalysts have the dimensions external diameter×internal diameter×length of (4-10 mm)×(2-8 mm)×(2-10 mm). Particularly preferred catalysts have the dimensions external diameter×internal diameter×length of (5-8 mm)×(3-5 mm)×(2-6 mm).

If the catalyst is a coated catalyst, the support body (a) preferably has the dimensions external diameter×height×internal diameter of (4-10 mm)×(2-8 mm)×(2-10 mm). The support body particularly preferably has the dimensions external diameter×height×internal diameter of (5-8 mm)×(3-5 mm)×(2-6 mm). The layer thickness D of the coating (b) composed of a multimetal oxide composition comprising molybdenum and at least one further metal is generally from 5 to 1000 μm. Preference is given to from 10 to 800 μm, particularly preferably from 50 to 600 μm and very particularly preferably from 80 to 500 μm.

Particularly preferred chromium(VI)-free catalytically active multimetal oxides comprising molybdenum and at least one further metal have the general formula (IIa):

Mo₁₂BiₐFe_{b}CO_{c}Ni_{d}CrₑX¹_{f}X²_{g}O_{y} (IIa),

where
X¹=Si, and/or Al,
X²=Li, Na, K, Cs and/or Rb,
0.2≤a≤1,
0.5≤b≤10,
0≤c≤10,
0≤d≤10,
2≤c+d≤10,
0.1≤e≤2,
0≤f≤10,
0≤g≤0.5,
y=a number determined by the valence and abundance of the elements other than oxygen in (IIa) in order to maintain electrical neutrality.

Preference is given to catalysts whose catalytically active oxide composition has, among the two metals Co and Ni, only Co (d=0). X¹ is preferably Si and X² is preferably K, Na and/or Cs, particularly preferably X²=K.

The stoichiometric coefficient a in the formula (IIa) is preferably such that 0.4≤a≤1, particularly preferably 0.4≤a≤0.95. The value of the variable b is preferably in the range 1≤b≤5 and particularly preferably in the range 2≤b≤4. The sum of the stoichiometric coefficient c+d is preferably in the range 4≤c+d≤8, and particularly preferably in the range 6≤c+d≤8. The stoichiometric coefficient e is preferably in the range 0.1≤e≤2, and particularly preferably in the range 0.2≤e≤1. The stoichiometric coefficient g is advantageously ≥0 Preference is given to 0.01≤g≤0.5 and particular preference is given to 0.05≤g≤0.2.

The value of the stoichiometric coefficient of oxygen, y, is determined by the valence and abundance of the cations in order to maintain electrical neutrality. Coated catalysts according to the invention having catalytically active oxide compositions whose molar ratio of Co/Ni is at least 2:1, preferably at least 3:1 and particularly preferably at least 4:1, are advantageous. It is best for only Co to be present.

The coated catalyst is produced by applying a layer comprising the chromium(VI)-free multimetal oxide comprising molybdenum, chromium and at least one further metal by means of a binder to the support body and drying and thermally treating the coated support body (coated catalyst precursor).

Production of the Catalyst

Production of the Multimetal Oxide Composition Comprising Molybdenum and a Further Metal

To produce suitable finely divided multimetal oxide compositions, known starting compounds for the elemental constituents other than oxygen of the desired multimetal oxide composition are used as starting materials in the respective stoichiometric ratio and a very intimate, preferably finely divided dry mixture is produced from these and is then subjected to a thermal treatment (calcination). The sources can either be oxides or compounds which can be converted into oxides by heating, at least in the presence of oxygen. Apart from the oxides, possible starting compounds are therefore halides, nitrates, formates, oxalates, acetates, carbonates or hydroxides, in particular.

Suitable starting compounds of molybdenum are the oxo compounds thereof (molybdates) or the acids derived from these.

Suitable starting compounds of Bi, Fe and Co are, in particular, the nitrates thereof.

The intimate mixing of the starting compounds can in principle be carried out in dry form or in the form of the aqueous solutions or suspensions.

An aqueous suspension can, for example, be produced by combining a solution comprising at least molybdenum and an aqueous solution comprising the remaining metals. Alkali metals or alkaline earth metals can be present in both solutions. Combining of the solutions results in a precipitation which leads to formation of a suspension. The temperature of the precipitation can be greater than room temperature, preferably from 30° C. to 95° C. and particularly preferably from 35° C. to 80° C. The suspension can then be aged at elevated temperature for a particular period of time. The aging time is generally in the range from 0 to 24 hours, preferably from 0 to 12 hours and particularly preferably from 0 to 8 hours. The temperature during aging is generally in the range from 20° C. to 99° C., preferably from 30° C. to 90° C. and particularly preferably from 35° C. to 80° C. During the precipitation and aging of the suspension, this is generally mixed by stirring. The pH of the mixed solutions or suspension is generally in the range from pH 1 to pH 12, preferably from pH 2 to pH 11 and particularly preferably from 15 pH 3 to pH 10.

Removal of the water produces a solid which represents an intimate mixture of the metal components introduced. The drying step can generally be carried out by evaporation, spray drying or freeze drying or the like. Drying is preferably carried out by spray drying. The suspension is for this purpose atomized at elevated temperature by means of a spraying head whose temperature is generally from 120° C. to 300° C. and the dried product is collected at a temperature of >60° C. The residual moisture content, determined by drying the spray powder at 120° C., is generally less than 20% by weight, preferably less than 15% by weight and particularly preferably less than 12% by weight.

Production of Shaped Bodies

The spray-dried powder is converted into a shaped body in a further step. Possible shaping aids (lubricants) are, for example, water, boron trifluoride or graphite. Based on the composition to be shaped to give the shaped body, it is usual to add ≤10% by weight, mostly ≤6% by weight, frequently ≤4% by weight, of shaping aid. The abovementioned amount added is normally >0.5% by weight. Graphite is the preferred lubricant according to the invention.

The calcination of the shaped bodies is generally carried out at temperatures above 350° C. However, a temperature of 650° C. is normally not exceeded during the thermal treatment. According to the invention, it is advantageous not to exceed a temperature of 650° C. and particularly preferably a temperature of 550° C. during the thermal treatment. Furthermore, a temperature of 380° C., advantageously a temperature of 400° C., particularly advantageously a temperature of 420° C. and very particularly preferably a temperature of 440° C., is preferably exceeded during the thermal treatment of the shaped body in the process of the invention. Here, the thermal treatment can be divided into a plurality of sections over time. For example, a thermal treatment can firstly be carried out at a temperature of from 150 to 350° C., preferably from 220 to 280° C., and a thermal treatment at a temperature of from 400 to 600° C., preferably from 430 to 550° C. can subsequently be carried out. The thermal treatment of the shaped body normally takes a number of hours (mostly more than 5 hours). The total time of the thermal treatment frequently extends to more than 10 hours. Treatment times of 45 hours or 35 hours are usually not exceeded during the thermal treatment of the shaped body. The total treatment time is often less than 30 hours. A temperature of 550° C. is preferably not exceeded during the thermal treatment of the shaped body and the treatment time in the temperature window of ≥400° C. preferably extends to from 5 to 30 hours.

The calcination of the shaped bodies can be carried out either under inert gas or under an oxidative atmosphere such as air (mixture of inert gas and oxygen) or else under a reducing atmosphere (e.g. a mixture of inert gas, NH₃, CO and/or H₂ or methane). Of course, the thermal treatment can also be carried out under reduced pressure. The thermal treatment of the shaped bodies can in principle be carried out in a wide variety of furnace types, e.g. heatable convection air chambers, tray furnaces, rotary tube furnaces, belt calciners or shaft furnaces. The thermal treatment of the shaped bodies is preferably carried out in a belt calciner apparatus as recommended in DE-A 10046957 and WO 02/24620. The thermal treatment of the shaped bodies below 350° C. generally results in thermal decomposition of the sources of the elemental constituents of the desired catalyst comprised in the shaped bodies. In the process of the invention, this decomposition phase frequently occurs during heating to temperatures of <350° C.

Milling of the Shaped Bodies

The calcined shaped body composed of multimetal oxide is milled in a further step to give multimetal oxide particles having a d₅₀ value of from 2 to 12 μm. The particle size distributions reported are based on a laser light scattering measurement on dry powders using a Metasizer 3000 from Malvern. Milling can be carried out using any suitable mill, e.g. opposed jet mills, rotor impingement mills, spiral jet mills or pin mills. The milling to this particle size is preferably carried out in opposed jet mills or rotor impingement mills having a static or dynamic classifier integrated into the mill.

In the case of opposed jet mills, the material to be introduced is conveyed by means of a metering screw and/or a lock into the milling space. The milling gas is introduced into the milling chamber via nozzles and depressurized in the process. The particles are accelerated in the region of the milling gas jets and impinge on one another at the focal point of the milling gas jets. They are comminuted here by means of particle-particle impacts. The milling gas flows together with the comminuted product material to the classifying wheel of the air classifier. There, separation into fines and coarse material occurs, as a function of the amount of gas and the circumferential velocity of the classifying wheel. The fines leave the milling space together with the milling gas via a discharge line and are precipitated in the downstream cyclone or filter. The coarse material goes back into the stressing zone and is comminuted further until the desired milling fineness has been achieved.

In the case of rotor impingement mills, the communition tool is a rotor which is equipped on the outer edge with beater plates. In the case of mills having a static classifier, the rotor is generally arranged horizontally, while in the case of mills having a dynamic classifier it is arranged vertically. It rotates at circumferential velocities of up to 120 m/s. The product to be milled is introduced centrally into the rotor, comminuted by the beater elements and then impinges on a milling track which concentrically encloses the rotor, with further communition taking place here. After repeated stressing, the gas stream drawn in by the mill transports the particles into the classifying zone. In the case of mills having a static classifier, this consists of an orifice plate located downstream of the rotor and a downstream blower wheel. A gas vortex is formed in front of the orifice plate. Centrifugal force and the entrainment force of the gas act on the particles which are rotating in a circular fashion. In the case of large particles, the entrainment force is higher and they go back into the milling zone, while in the case of finer particles, the entrainment force is greater. The latter leave the mill together with the gas stream through the orifice plate as milled material. In modern mills having a dynamic classifier, classification takes place not in a gas vortex but in a classifier wheel. This generally consists of a rotating wheel in which intermediate spaces are located between webs. The particles transported by the milling gas between the laminae are accelerated on a circular track and separated by centrifugal and entrainment forces. The rejection wheel makes a significantly sharper separation possible than the static classifier. The average fineness and the steepness of the particle size distribution are influenced essentially by the following parameters: circumferential velocity (rotational speed) of the milling rotor, number and type of the beater elements, type of milling track, centrifugal acceleration (rotational speed) of the classifier wheel/orifice plate diameter, gas throughput, product throughput.

Mixing of the Pulverulent Multimetal Oxide with Chromium(III) Oxide

In a further step, the pulverulent multimetal oxide is mixed with chromium(III) oxide. Here, it is possible to use any suitable mixers. Mixing is preferably carried out using plowshare mixers, e.g. from Lödige. These mixers operate according to the principle of a mechanically generated fluidized bed: the plowshare blades rotate in the vicinity of the wall in a horizontal cylindrical drum. These blades lift the mixing components from the drum wall and fling them from the bed of material into the free mixing space. With continual catching of the entire product, intensive mixing is achieved in this way. The optional installation of knife heads is omitted in a preferred variant. In a preferred variant, from 10 to 15 g of chromium(III) oxide are added per kg of milled pulverulent multimetal oxide, with the rotating drum being filled to a degree of from 55 to 65%. The rotational speed v of the rotating drum is selected so that a Froude number Fr of from 0.5 to 5, preferably from 2.8 to 3.2, is obtained, according to:

$$Fr = \frac{{v}^{2}}{r \cdot g} ,$$

where r is the radius of the rotating drum and g is the acceleration due to gravity,

Thermal Treatment of the Pulverulent Mixture of Multimetal Oxide and Chromium(III) Oxide

In a further step, the mixture is subjected to a solid-state reaction by means of a thermal treatment. The thermal treatment of the mixture is generally carried out at temperatures above 350° C. However, a temperature of 650° C. is normally not exceeded during the thermal treatment. According to the invention, a temperature of 600° C., preferably a temperature of 550° C. and particularly preferably a temperature of 530° C., is advantageously not exceeded during the thermal treatment. Furthermore, a temperature of 390° C., advantageously a temperature of 450° C., particularly advantageously a temperature of 470° C., is preferably exceeded during the thermal treatment of the mixture in the process of the invention. Here, the thermal treatment can be divided into a number of sections over time. In particular, there is typically a heating phase commencing at room temperature, a phase in which the target temperature is kept approximately constant (plateau) and a cooling phase. The thermal treatment of the mixture normally takes a number of hours (mostly more than 5 hours). The total time of the thermal treatment frequently extends to more than 7 hours. Treatment times of 24 h are usually not exceeded during the thermal treatment of the mixture. The temperature preferably rises along a linear ramp from room temperature to the target temperature over a period of from 3 to 4 hours during the heating phase. The treatment time in the temperature window of ≥390° C. extends from 1 to 16 hours, preferably from 3 to 8 hours and particularly preferably from 3 to 4 hours. The thermal treatment can be carried out under either inert gas or under an oxidative atmosphere such as air (mixture of inert gas and oxygen) or else under a reducing atmosphere (e.g. mixture of inert gas, NH₃, CO and/or H₂ or methane). Of course, the thermal treatment can also be carried out under reduced pressure. The thermal treatment of the mixture can in principle be carried out in a wide variety of furnace types, e.g. electrically heatable or fired chamber furnaces, tray furnaces, rotary tube furnaces, belt calciners, shuttle kilns, hood furnaces or shaft furnaces. The thermal treatment of the mixture is preferably carried out in chamber furnaces which are fired directly by means of natural gas and can be charged by means of saggars. The desired temperature can be set by control of the secondary air.

Production of Coated Catalysts

The pulverulent multimetal oxide composition can additionally be applied with the aid of a liquid binder to the outer surface of a support body in order to produce a coated catalyst. The fineness of the catalytically active oxide composition to be applied to the surface of the support body will of course be adapted in accordance with the desired coating thickness.

Support materials suitable for coated catalysts according to the invention are porous or preferably nonporous aluminum oxides, silicon dioxide, zirconium dioxide, silicon carbide or silicates such as magnesium silicate or aluminum silicate. The materials of the support bodies are chemically inert.

The support materials can be porous or nonporous. The support material is preferably nonporous (total volume of the pores, based on the volume of the support body, preferably ≤1% by volume).

Preferred hollow cylinders as support bodies have a length of from 2 to 10 mm and an external diameter of from 4 to 10 mm. Furthermore, the wall thickness is preferably from 1 to 4 mm. Particularly preferred annular support bodies have a length of from 2 to 6 mm, an external diameter of from 5 to 8 mm and a wall thickness of from 1 to 2 mm. An example is rings having the geometry 7 mm×4 mm×3 mm (external diameter×internal diameter×length) as support bodies. A preferred support composed of steatite of the C 220 type is marketed, for example, by CeramTec. Preferred supports are also described in DE 10 2008 001402 and WO 09/133065.

The layer thickness D of a multimetal oxide composition comprising molybdenum, chromium and at least one further metal is generally from 5 to 1000 μm. Preference is given to from 10 to 800 μm, particularly preferably from 50 to 600 μm and very particularly preferably from 80 to 500 μm.

The application of the multimetal oxide comprising molybdenum, chromium and at least one further metal to the surface of the support body can be carried out in a manner corresponding to the processes described in the prior art, for example as in US-A 2006/0205978 and EP-A 0 714 700.

In general, the finely divided compositions are applied to the surface of the support body with the aid of a liquid binder. Possible liquid binders are, for example, water, an organic solvent or a solution of an organic substance (e.g. an organic solvent) in water or in an organic solvent.

It is particularly advantageous to use a solution consisting of from 20 to 95% by weight of water and from 5 to 80% by weight of an organic compound as liquid binder. The proportion of organics in the abovementioned liquid binders is preferably from 5 to 50% by weight and particularly preferably from 8 to 30% by weight.

Preference is given to organic binders or binder components whose boiling point or sublimination temperature at atmospheric pressure (1 atm) is ≥100° C., preferably ≥150° C. The boiling point or sublimination point of such organic binders or binder components at atmospheric pressure is very particularly preferably also below the maximum calcination temperature employed in the production of the finely divided molybdenum-comprising multimetal oxide. This maximum calcination temperature is usually ≤600° C., frequently ≤550° C.

Particularly preferred liquid binders are solutions consisting of from 20 to 95% by weight of water and from 5 to 80% by weight of glycerol. The proportion of glycerol in these aqueous solutions is preferably from 5 to 50% by weight and particularly preferably from 8 to 35% by weight.

The application of the finely divided multimetal oxide comprising molybdenum and chromium can be carried out by dispersing the finely divided multimetal oxide composition comprising molybdenum and chromium in the liquid binder and spraying the resulting suspension onto agitated and optionally hot support bodies, as described in DE-A 1 642 921, and DE-A 2 626 887. After spraying-on is complete, it is possible, as described in DE-A 2 909 670, to reduce the moisture content of the resulting coated catalysts by passing hot air over them.

However, preference is given to firstly moistening the support bodies with the liquid binder and subsequently applying the finely divided multimetal oxide composition to the surface of the support body moistened with binder by rolling the moistened support bodies in the finely divided composition. To achieve the desired layer thickness, the above-described process is preferably repeated a number of times, i.e. the initially coated support body is again moistened and then coated by contact with dry finely divided composition.

To carry out the process on an industrial scale, it is advisable to employ the process disclosed in DE-A 2 909 671, but preferably using the binders recommended in EP-A 714 700. That is to say, the support bodies to be coated are introduced into a preferably inclined (the angle of inclination is generally from 30 to 90°) rotating vessel (e.g. rotary plate or coating drum). The rotating vessel conveys the hollow cylindrical support bodies under two metering devices which are arranged successively at a particular spacing. The first of the two metering devices is advantageously a nozzle by means of which the support bodies rolling in the rotating plate are sprayed with the liquid binder to be used and moistened in a controlled manner. The second metering device is located outside the spray cone of the liquid binder sprayed in and serves to introduce the finely divided composition, for example via a vibratory chute. The support bodies which have been moistened in a controlled manner take up the introduced active composition powder which is compacted by the rolling motion on the outer surface of the cylindrical support bodies to form a cohesive coating.

If required, the support body which has been initially coated in this way once again travels through under the spray nozzle during the subsequent rotation and is thus moistened in a controlled manner in order to be able to take up a further layer of finely divided composition during its further motion. Intermediate drying is generally not necessary. Removal of the liquid binder can be effected, partly or completely, by final supply of heat, e.g. by the action of hot gases such as N₂ or air. A particular advantage of the above-described embodiment of the process is that coated catalysts having coatings consisting of two or more different compositions arranged in layers can be produced in one operation. It is remarkable that the process results in both fully satisfactory adhesion of the subsequent layers to one another and also to the initial coat on the surface of the support body. This also applies in the case of annular support bodies.

The temperatures necessary to bring about removal of the bonding agent are below the maximum calcinations temperature of the catalyst, generally in the range from 200° C. to 600° C. In order to remove the bonding agent, the catalyst can be heated to from 240° C. to 500° C. and particularly preferably to temperatures in the range from 260° C. to 400° C. The time required for removal of the bonding agent can be a number of hours. The catalyst can be heated to the temperature indicated for from 0.5 to 24 hours in order to remove the bonding agent. The time is preferably in the range from 1.5 to 8 hours and particularly preferably in the range from 2 to 6 hours. Passing a gas around the catalyst can accelerate the removal of the bonding agent. The gas is preferably air or nitrogen and particularly preferably air. The removal of the bonding agent can, for example, be carried out in an oven through which gas flows or in a suitable drying apparatus, or for example in a belt dryer. However, the bonding agent is preferably firstly burnt off in the reactor before the actual reaction, particularly preferably during the start-up phase of the catalyst, where the start-up phase preferably comprises the steps:

i) introduction of an oxygen-comprising gas stream and an inert gas stream into the dehydrogenation zone in such a ratio that the oxygen content of the gas stream fed in corresponds to 80% of the oxygen content of the corresponding gas stream in the operating phase;

ii) setting of the gas flow fed in to at least 70% of the volume flow of the corresponding gas stream in the operating phase;

iii) optional introduction, in the case of an initial oxygen content of the gas stream fed in of from 30 to 80% of the oxygen content of the corresponding gas stream in the operating phase, of a stream of steam into the dehydrogenation zone;

iv) introduction, in the case of an initial oxygen content of the gas stream fed in of from 30 to 80% of the oxygen content of the corresponding gas stream in the operating phase, of an oxygen-comprising gas stream a2′ and a butene-comprising feed gas stream a1′ having lower volume flows than in the operating phase in a ratio of k=a2′/a1′, and increasing of the volume flows of the gas streams a1′ and a2′ until the volume flows of the gas streams a1 and a2 in the operating phase have been attained, where the gas stream fed in is at least 70% and not more than 120% of the volume flow in the operating phase.

In general, the pressure in the dehydrogenation zone during the start-up phase is from 1 to 5 bar absolute, preferably from 1.05 to 2.5 bar. In general, the temperature of the heat transfer medium during the start-up phase is in the range from 220 to 490° C., preferably from 300 to 450° C. and particularly preferably from 330 to 420° C. In general, the duration of the start-up phase is in the range from 1 to 7200 minutes, preferably from 5 to 2000 minutes and particularly preferably from 10 to 500 minutes.

If the bonding agent is removed during the start-up phase, the coating closes on having hot air passed over it in order to reduce the moisture content of the resulting coated catalysts, as described in DE-A 2 909 670.

It has surprisingly been found that the catalyst of the invention is more active and more selective when the start-up phase comprises relatively long periods of time of from 2000 to 7200 minutes. In addition, the catalyst of the invention is more active and more selective after one or more further regeneration phases analogous to the first start-up phase (see also FIG. 2). For this reason, regeneration of the catalyst of the invention in a manner analogous to the start-up phase is useful after a certain interval, preferably after from 5 to 15 days of operation.

Oxidative Dehydrogenation (Oxydehydrogenation, ODH)

The present invention also provides for the use of the all-active catalysts and coated catalysts of the invention in a process for the oxidative dehydrogenation of 1-butene and/or 2-butene to butadiene. The catalysts of the invention display a high activity and in particular also a high selectivity for the formation of 1,3-butadiene from 1-butene and 2-butene.

The invention also provides a process for the oxidative dehydrogenation of n-butenes to butadiene, wherein a starting gas mixture comprising n-butenes is mixed with an oxygen-comprising gas and optionally additional inert gas or steam and is brought into contact with the catalyst arranged in a fixed catalyst bed at a temperature of from 220 to 490° C. in a fixed-bed reactor.

The reaction temperature of the oxydehydrogenation is generally controlled by means of a heat transfer medium which is present around the reaction tubes. Possible liquid heat transfer media of this type are, for example, melts of salts such as potassium nitrate, potassium nitrite, sodium nitrite and/or sodium nitrate and also melts of metals such as sodium, mercury and alloys of various metals. However, ionic liquids or heat transfer oils can also be used. The temperature of the heat transfer medium is in the range from 220 to 490° C. and preferably from 300 to 450° C. and particularly preferably from 350 to 420° C.

Owing to the exothermic nature of the reactions which proceed, the temperature in particular sections of the interior of the reactor during the reaction can be higher than that of the heat transfer medium and a hot spot is formed. The position and height of the hot spot are determined by the reaction conditions, but they can also be regulated by means of the dilution ratio of the catalyst bed or the flow of mixed gas. The difference between hot spot temperature and the temperature of the heat transfer medium is generally in the range from 1 to 150° C., preferably from 10 to 100° C. and particularly preferably from 20 to 80° C. The temperature at the end of the catalyst bed is generally from 0 to 100° C. higher, preferably from 0.1 to 50° C. higher, particularly preferably from 1 to 25° C. higher, than the temperature of the heat transfer medium.

The oxydehydrogenation can be carried out in all fixed-bed reactors known from the prior art, for example in a tray oven, in a fixed-bed tube reactor or shell-and-tube reactor or in a plate heat exchanger reactor. A shell-and-tube reactor is preferred.

The oxidative dehydrogenation is preferably carried out using the catalysts of the invention in fixed-bed tube reactors or fixed-bed shell-and-tube reactors. The reaction tubes are, like the other elements of the shell-and-tube reactor, generally made of steel. The wall thickness of the reaction tubes is typically from 1 to 3 mm. Their internal diameter is generally (uniformly) from 10 to 50 mm or from 15 to 40 mm, frequently from 20 to 30 mm. The number of reaction tubes installed in the shell-and-tube reactor is generally at least 1000, or 3000, or 5000, preferably at least 10 000. The number of reaction tubes installed in the shell-and-tube reactor is frequently from 15 000 to 30 000 or up to 40 000 or up to 50 000. Shell-and-tube reactors having a number of reaction tubes above 50 000 tend to be the exception.

The length of the reaction tubes is normally a few meters; a reaction tube length in the range from 1 to 8 m, frequently from 2 to 7 m, often from 2.5 to 6 m, is typical.

Among the reaction tubes, a distinction is normally made between working tubes and thermo tubes. While the working tubes are those reaction tubes in which the partial oxidation to be carried out is actually carried out, thermo tubes have the primary purpose of, standing in for all working tubes, monitoring and controlling the reaction temperature along the reaction tubes. For this purpose, the thermo tubes normally comprise, in addition to the fixed catalyst bed, a temperature sensor sheath which is equipped only with a temperature sensor and is located centered along the axis of the thermo tube. In general, the number of thermo tubes in a shell-and-tube reactor is very much smaller than the number of the working tubes. The number of thermo tubes is normally ≤20.

Furthermore, the catalyst installed in the reactor can, as described above, consist of a single zone or two or more zones. These zones can consist of pure catalyst or be diluted with a material which does not react with the starting gas or components of the product gas from the reaction. Furthermore, the catalyst zones can consist of all-active catalysts or supported coated catalysts.

As starting gas, it is possible to use pure n-butenes (1-butene and/or cis-/trans-2-butene) or else a gas mixture comprising butenes. Such a gas mixture can be obtained, for example, by nonoxidative dehydrogenation of n-butane. It is also possible to use a fraction which comprises n-butenes (1-butene and/or 2-butene) as main constituent and has been obtained from the C₄ fraction from the cracking of naphtha by removal of butadiene and isobutene. Furthermore, gas mixtures which comprise pure 1-butene, cis-2-butene, trans-2-butene or mixtures thereof and have been obtained by dimerization of ethylene can also be used as starting gas. Furthermore, gas mixtures which comprise n-butenes and have been obtained by fluid catalytic cracking (FCC) can be used as starting gas. In one embodiment of the process of the invention, the starting gas mixture comprising n-butenes is obtained by nonoxidative dehydrogenation of n-butane. The coupling of a nonoxidative catalytic dehydrogenation with the oxidative dehydrogenation of the n-butenes formed makes it possible to obtain a high yield of butadiene, based on n-butane used.

The nonoxidative catalytic dehydrogenation of n-butane gives a gas mixture comprising not only butadiene but also 1-butene, 2-butene and unreacted n-butane and secondary constituents. Usual secondary constituents are hydrogen, water vapor, nitrogen, Co and CO₂, methane, ethane, ethene, propane and propene. The composition of the gas mixture leaving the first dehydrogenation zone can vary greatly as a function of the conditions under which the dehydrogenation is carried out. Thus, when the dehydrogenation is carried out with introduction of oxygen and additional hydrogen, the product gas mixture has a comparatively high content of water vapor and carbon oxides. In modes of operation without introduction of oxygen, the product gas mixture from the nonoxidative dehydrogenation has a comparatively high content of hydrogen.

The product gas stream from the nonoxidative dehydrogenation of n-butane typically comprises from 0.1 to 15% by volume of butadiene, from 1 to 15% by volume of 1-butene, from 1 to 25% by volume of 2-butene (cis/trans-2-butene), from 20 to 70% by volume of n-butane, from 1 to 70% by weight of water vapor, from 0 to 10% by volume of low-boiling hydrocarbons (methane, ethane, ethene, propane and propene), from 0.1 to 40% by volume of hydrogen, from 0 to 70% by volume of nitrogen and from 0 to 5% by volume of carbon oxides.

The product gas stream from the nonoxidative dehydrogenation can be fed without further work-up to the oxidative dehydrogenation.

Furthermore, any impurities can be present in the starting gas for the oxydehydrogenation in a range in which the operation of the present invention is not inhibited. In the case of the preparation of butadiene from n-butenes (1-butene and cis-/trans-2-butene), mention may be made of saturated and unsaturated, branched and unbranched hydrocarbons such as methane, ethane, ethene, acetylene, propane, propene, propyne, n-butane, isobutane, isobutene, n-pentane and also dienes such as 1,2-butadiene as impurities. The amounts of impurities are generally 70% or less, preferably 30% or less, more preferably 10% or less and particularly preferably 1% or less. The concentration of linear monoolefins having 4 or more carbon atoms (n-butenes and higher homologs) in the starting gas is not subject to any particular restrictions; it is generally in the range from 35.00 to 99.99% by volume, preferably from 71.00 to 99.0% by volume and even more preferably from 75.00 to 95.0% by volume.

To carry out the oxidative dehydrogenation with complete conversion of butenes, it is necessary to use a gas mixture which has a molar oxygen: n-butenes ratio of at least 0.5. Preference is given to working at an oxygen: n-butenes ratio of from 0.55 to 10. To set this value, the starting material gas can be mixed with oxygen or an oxygen-comprising gas, for example air, and optionally additional inert gas or steam. The oxygen-comprising gas mixture obtained is then fed to the oxydehydrogenation.

The gas comprising molecular oxygen which is used according to the invention is a gas which generally comprises more than 10% by volume, preferably more than 15% by volume and even more preferably more than 20% by volume, of molecular oxygen and specifically is preferably air. The upper limit for the content of molecular oxygen is generally 50% by volume or less, preferably 30% by volume or less and even more preferably 25% by volume or less. In addition, any inert gases can be present in the gas comprising molecular oxygen in a range in which the operation of the present invention is not inhibited. As possible inert gases, mention may be made of nitrogen, argon, neon, helium, CO, CO₂ and water. The amount of inert gases is in the case of nitrogen generally 90% by volume or less, preferably 85% by volume or less and even more preferably 80% by volume or less. In the case of constituents other than nitrogen, it is generally 10% by volume or less, preferably 1% by volume or less. If this amount becomes too great, it becomes ever more difficult to supply the reaction with the necessary oxygen.

Furthermore, inert gases such as nitrogen and also water (in the form of water vapor) can be comprised together with the mixed gas composed of starting gas and the gas comprising molecular oxygen. Nitrogen is present in order to adjust the oxygen concentration and to prevent formation of an explosive gas mixture, and the same applies to steam. Steam is also present in order to control carbonization of the catalyst and to remove the heat of reaction. Preference is given to mixing water (as steam) and nitrogen into the mixed gas and introducing the latter into the reactor. When steam is introduced into the reactor, preference is given to introducing a proportion of from 0.2 to 5.0 (proportion by volume), preferably from 0.5 to 4 and even more preferably from 0.8 to 2.5, based on the amount of the abovementioned starting gas introduced. When nitrogen gas is introduced into the reactor, preference is given to introducing a proportion of from 0.1 to 8.0 (proportion by volume), preferably from 0.5 to 5.0 and even more preferably from 0.8 to 3.0, based on the amount of the abovementioned starting gas introduced.

The proportion of the starting gas comprising the hydrocarbons in the mixed gas is generally 4.0% by volume or more, preferably 6.0% by volume or more and even more preferably 8.0% by volume or more. On the other hand, the upper limit is 20% by volume or less, preferably 16.0% by volume or less and even more preferably 13.0% by volume or less. To reliably avoid the formation of explosive gas mixtures, nitrogen gas is firstly introduced into the starting gas or into the gas comprising molecular oxygen before the mixed gas is obtained, the starting gas and the gas comprising molecular oxygen are mixed to give the mixed gas and this mixed gas is then preferably introduced.

During steady-state operation, the residence time in the reactor is not subject to any particular restrictions according to the present invention, but the lower limit is generally 0.3 s or more, preferably 0.7 s or more and even more preferably 1.0 s or more. The upper limit is 5.0 s or less, preferably 3.5 s or less and even more preferably 2.5 s or less. The ratio of the flow of mixed gas to the amount of catalyst in the reactor interior is from 500 to 8000 h⁻¹, preferably from 800 to 4000 h⁻¹ and even more preferably from 1200 to 3500 h⁻¹. The WHSV of butenes over the catalyst (expressed in g_{butenes}/(g_{catalyst}*hour)) in steady-state operation is generally 0.1-5.0 h⁻¹, preferably 0.2-3.0 h⁻¹, and even more preferably from 0.25 to 1.0 h⁻¹. The volume and mass of the catalyst relate to the complete catalyst consisting of support and active composition.

The volume change factor describes the flow difference from reactor inlet to outlet and is dependent on the flow of starting gas at the reactor inlet and on the flow of product gas at the reactor outlet. It can advantageously be determined via the ratio of the volume concentration of an inert constituent, i.e. a constituent which is not in any form reacted in the reactor (for example Ar or N2), of the reaction gas at the reactor inlet and the reactor outlet. The volume change factor can be from 1 to 1.15, preferably from 1 to 1.1 and particularly preferably from 1.01 to 1.08.

Work-Up of the Product Gas Stream

The product gas stream leaving the oxidative dehydrogenation generally comprises not only butadiene but also unreacted n-butane and isobutane, 2-butene and water vapor. As secondary constituents, it generally comprises carbon monoxide, carbon dioxide, oxygen, nitrogen, methane, ethane, ethene, propane and propene, possibly hydrogen and also oxygen-comprising hydrocarbons, known as oxygenates. In general, it comprises only small proportions of 1-butene and isobutene.

For example, the product gas stream leaving the oxidative dehydrogenation can comprise from 1 to 40% by volume of butadiene, from 20 to 80% by volume of n-butane, from 0 to 5% by volume of isobutane, from 0.5 to 40% by volume of 2-butene, from 0 to 5% by volume of 1-butene, from 0 to 70% by volume of water vapor, from 0 to 10% by volume of low-boiling hydrocarbons (methane, ethane, ethene, propane and propene), from 0 to 40% by volume of hydrogen, from 0 to 30% by volume of oxygen, from 0 to 70% by volume of nitrogen, from 0 to 10% by volume of carbon oxides and from 0 to 10% by volume of oxygenates. Oxygenates can be, for example, formaldehyde, furan, acetic acid, maleic anhydride, formic acid, methacrolein, methacrylic acid, crotonaldehyde, crotonic acid, propionic acid, acrylic acid, methyl vinyl ketone, styrene, benzaldehyde, benzoic acid, phthalic anhydride, fluorenone, anthraquinone and butyraldehyde.

Some of the oxygenates can oligomerize further and dehydrogenate on the catalyst surface and during the work-up and thus form deposits comprising carbon, hydrogen and oxygen, hereinafter referred to as carbonaceous material. These deposits can lead to interruptions to the operation of the process for the purpose of cleaning and regeneration and are therefore undesirable. Typical carbonaceous material precursors are styrene, fluorenone and anthraquinone. For this reason, it is useful to carry out a regeneration of the catalyst of the invention in a manner analogous to the start-up phase after a certain interval, preferably after from 5 to 15 days of operation. It has surprisingly been found that the catalyst of the invention is generally more active and more selective after the regeneration compared to the selectivity and activity after the first start-up phase.

A major part of the high-boiling secondary component and of the water can subsequently be separated off from the product gas stream by cooling. This separation is preferably effected in a quench. This quench can consist of one or more stages. Preference is given to using a process in which the product gas is brought into direct contact with the cooling medium and is thereby cooled. The cooling medium is not subjected to any particular restrictions, but preference is given to using water or an alkali aqueous solution.

Preference is given to a two-stage quench. The cooling temperature of the product gas differs as a function of the temperature of the product gas obtained from the reactor outlet and of the cooling medium. In general, the product gas can, depending on the presence and temperature level of a heat exchanger, obtain a temperature of from 100 to 440° C., preferably from 140 to 300° C., in particular from 170 to 240° C., before the inlet into the quench. The product gas inlet into the quench has to be designed so that blockage caused by deposits at and directly before the gas inlet is minimized or prevented. In the 1^{st} quenching stage, the product gas is brought into contact with the cooling medium. Here, the cooling medium can be introduced through a nozzle in order to achieve very efficient mixing with the product gas. For the same purpose, internals such as further nozzles through which the product gas and the cooling medium have to pass together can be installed in the quenching stage. The coolant inlet into the quench has to be designed so that blockage caused by deposits in the region of the coolant inlet is minimized or prevented.

In general, the product gas is cooled to from 5 to 180° C., preferably to from 30 to 130° C. and even more preferably to from 60 to 90° C., in the first quenching stage. The temperature of the cooling medium at the inlet can generally be from 25 to 200° C., preferably from 40 to 120° C., in particular from 50 to 90° C. The pressure in the first quenching stage is not subject to any particular restrictions but is generally from 0.01 to 4 bar (gauge), preferably from 0.1 to 2 bar (gauge) and particularly preferably from 0.2 to 1 bar (gauge). When many high-boiling by-products are present in the product gas, polymerization among the high-boiling by-products or deposits of solid by-products caused by high-boiling by-products in this process step easily occur. The cooling medium used in the cooling tower is frequently circulated so that blockages caused by solid precipitates can occur when the preparation of conjugated dienes is continued for a long time. The circulatory flow of the cooling medium in liters per hour based on the mass flow of butadiene in gram per hour can generally be from 0.0001 to 5 l/g, preferably from 0.001 to 1 l/g and particularly preferably from 0.002 to 0.2 l/g.

The dissolution of by-products of the ODH reaction, for example acetic acid, maleic anhydride, etc., in a cooling medium such as water occurs more readily at an elevated pH than at a low pH. Since the dissolution of by-products decreases the abovementioned pH of, for example, water, the pH can be kept constant or increased by addition of an alkaline medium. In general, the pH at the bottom of the first quenching stage is kept in the range from 2 to 14, preferably from 3 to 13, particularly preferably from 4 to 12. The more acidic the value, the less alkaline medium has to be introduced. The more basic, the more readily does the dissolution of some by-products occur. However, very high pH values lead to dissolution of by-products such as CO₂ and thus to a very high consumption of the alkaline medium. The temperature of the cooling medium in the bottom can generally be from 27 to 210° C., preferably from 45 to 130° C., in particular from 55 to 95° C. Since the loading of the cooling medium with secondary components increases over time, part of the loaded cooling medium can be taken off from the circuit and the amount circulated can be kept constant by addition of cooling medium which is not loaded. The ratio of amount discharged and amount added depends on the vapor loading of the product gas and the product gas temperature at the end of the first quenching stage. When the cooling medium is water, the amount added in the first quenching stage is generally lower than the amount discharged.

The cooled product gas stream which has been depleted in secondary components can then be fed to a second quenching stage. In this, it can once again be brought into contact with a cooling medium.

In general, the product gas is cooled to from 5 to 100° C., preferably to from 15 to 85° C. and even more preferably to from 30 to 70° C., up to the gas outlet from the second quenching stage. The coolant can be introduced in countercurrent to the product gas. In this case, the temperature of the cooling medium at the coolant inlet can be from 5 to 100° C., preferably from 15 to 85° C., in particular from 30 to 70° C. The pressure in the second quenching stage is not subject to any particular restrictions but is generally from 0.01 to 4 bar (gauge), preferably from 0.1 to 2 bar (gauge) and particularly preferably from 0.2 to 1 bar (gauge). The cooling medium used in the cooling tower is frequently circulated so that blockages caused by solid precipitates can occur when the preparation of conjugated dienes is continued for a long time. The circulatory flow of the cooling medium in liters per hour based on the mass flow of butadiene in gram per hour can generally be from 0.0001 to 5 l/g, preferably from 0.0001 to 1 l/g and particularly preferably from 0.002 to 0.2 l/g.

To be able to achieve very good contact between product gas and cooling medium, internals can be present in the second quenching stage. Such internals comprise, for example, bubble cap trays, centrifugal trays and/or sieve trays, columns having structured packings, e.g. sheet metal packings having a specific surface area of from 100 to 1000 m²/m³, e.g. Mellapak® 250 Y, and columns packed with random packing elements.

The circulations of the two quenching stages can either be separate from one another or be connected to one another. The desired temperature of the circulating streams can be set by means of suitable heat exchangers.

To minimize the entrainment of liquid constituents from the quench into the offgas line, it is possible to undertake suitable structural measures, for example the installation of a demister. Furthermore, high-boiling components which are not separated from the product gas in the quench can be removed from the product gas by means of further structural measures, for example gas scrubs. This gives a gas stream in which n-butane, 1-butene, 2-butenes, butadiene, possibly oxygen, hydrogen, water vapor, small amounts of methane, ethane, ethene, propane and propene, isobutane, carbon oxides and inert gases remain. Furthermore, traces of high-boiling components which are not separated off quantitatively in the quench can remain in this product gas stream.

The product gas stream from the quench is subsequently compressed in at least one first compression stage and subsequently cooled, with at least one condensate stream comprising water being condensed out and a gas stream comprising n-butane, 1-butene, 2-butenes, butadiene, possibly hydrogen, water vapor, small amounts of methane, ethane, ethene, propane and propene, isobutane, carbon oxides and inert gases, possibly oxygen and hydrogen remaining. The compression can be carried out in one or more stages. In total, the product gas is compressed from a pressure in the range from 1.0 to 4.0 bar (absolute) to a pressure in the range from 3.5 to 20 bar (absolute). Each compression stage is followed by a cooling stage in which the gas stream is cooled to a temperature in the range from 15 to 60° C. The condensate stream can thus also comprise a plurality of streams in the case of multistage compression.

The condensate stream generally comprises at least 80% by weight, preferably at least 90% by weight, of water and additionally comprising small amounts of low boilers, C₄-hydrocarbons, oxygenates and carbon oxides.

Suitable compressors are, for example, turbocompressors, rotary system compressors and reciprocating compressors. The compressors can, for example, be driven by an electric motor, an expander or a gas or steam turbine. Typical compression ratios (exit pressure: entry pressure) per compressor stage are, depending on the construction type, in the range from 1.5 to 3.0. Cooling of the compressed gas is effected by means of heat exchangers which can, for example, be configured as shell-and-tube heat exchangers, helically coiled heat exchangers or plate heat exchangers. Cooling water or heat transfer oils are used as coolants in the heat exchangers. In addition, air cooling using blowers is preferably employed.

The stream comprising butadiene, butenes, butane, inert gases and possibly carbon oxides, oxygen, hydrogen and low-boiling hydrocarbons (methane, ethane, ethene, propane, propene) and small amounts of oxygenates is passed as starting stream to the further work-up.

The removal of the low-boiling secondary constituents from the product gas stream can be effected by means of conventional separation processes such as distillation, membrane processes, absorption or adsorption.

In a preferred embodiment of the process, the incondensable or low-boiling gas constituents such as hydrogen, oxygen, carbon oxides, the low-boiling hydrocarbons (methane, ethane, ethene, propane, propene) and inert gas such as possibly nitrogen are separated off by means of a high-boiling absorption medium in an absorption/desorption cycle to give a C₄ product gas stream which consists essentially of the C₄-hydrocarbons. In general, the C₄-product gas stream comprises at least 80% by volume, preferably at least 90% by volume, particularly preferably at least 95% by volume, of the C₄-hydrocarbons, essentially n-butane, 2-butene and butadiene.

For this purpose, the product gas stream is, after prior removal of water, brought into contact with an inert absorption medium in an absorption stage and the C₄-hydrocarbons are absorbed in the inert absorption medium to give absorption medium loaded with C₄-hydrocarbons and an offgas comprising the remaining gas constituents. In a desorption stage, the C₄-hydrocarbons are liberated again from the absorption medium.

The absorption stage can be carried out in any suitable absorption column known to those skilled in the art. Absorption can be effected by simple passage of the product gas stream through the absorption medium. However, it can also be carried out in columns or in rotary absorbers. The absorption can be carried out in cocurrent, countercurrent or cross-current. The absorption is preferably carried out in countercurrent. Suitable absorption columns are, for example, tray columns having bubble cap trays, centrifugal trays and/or sieve trays, columns having structured packings, e.g. sheet metal packings having a specific surface area of from 100 to 1000 m²/m³, e.g. Mellapak® 250 Y, and columns packed with random packing elements. However, trickle towers and spray towers, graphite block absorbers, surface absorbers such as thick film and thin film absorbers and also rotational columns, plate scrubbers, crossed spray scrubbers and rotary scrubbers are also possible.

In one embodiment, the stream comprising butadiene, butene, butane, and/or nitrogen and possibly oxygen, hydrogen and/or carbon dioxide is fed into the lower region of an absorption column. In the upper region of the absorption column, the solvent and optionally water-comprising stream are introduced.

Inert absorption media used in the absorption stage are generally high-boiling nonpolar solvents in which the C₄-hydrocarbon mixture to be separated off has a significantly higher solubility than the remaining gas constituents to be separated off. In a preferred embodiment, an alkane mixture such as tetradecane (industrial C₁₄-C₁₇ fraction) is used as solvent for the absorption.

At the top of the absorption column, an offgas stream which comprises essentially inert gas, carbon oxides, possibly butane, butenes such as 2-butenes and butadiene, possibly oxygen, hydrogen and low-boiling hydrocarbons (for example methane, ethane, ethene, propane, propene) and water vapor is taken off. This stream can be partly fed to the ODH reactor or the O₂ removal reactor. This allows, for example, the feed stream to the ODH reactor to be set to the desired C₄-hydrocarbon content.

The solvent stream loaded with C₄-hydrocarbons is introduced into a desorption column. According to the invention, all column internals known to those skilled in the art are suitable for this purpose. In one process variant, the desorption step is carried out by depressurization and/or heating of the loaded solvent. A preferred process variant is the addition of stripping steam and/or the introduction of fresh steam in the bottom of the desorber. The solvent which has been depleted in C₄-hydrocarbons can be fed as a mixture together with the condensed steam (water) to a phase separation, so that the water is separated off from the solvent. All apparatuses known to those skilled in the art are suitable for this purpose. In addition, it is possible to utilize the water separated off from the solvent for generating the stripping steam.

Preference is given to using 70 to 100% by weight of solvent and from 0 to 30% by weight of water, particularly preferably from 80 to 100% by weight of solvent and from 0 to 20% by weight of water, in particular from 85 to 95% by weight of solvent and from 5 to 15% by weight of water. The absorption medium which has been regenerated in the desorption stage is recirculated to the absorption stage.

The separation is generally not quite complete, so that small amounts or even only traces of the further gas constituents, in particular the high-boiling hydrocarbons, can be present in the C₄ product gas stream, depending on the way in which the separation is carried out. The volume flow reduction also brought about by the separation reduces the load on the subsequent process steps.

The C₄ product gas stream consisting essentially of n-butane, butenes such as 2-butenes and butadiene generally comprises from 20 to 80% by volume of butadiene, from 20 to 80% by volume of n-butane, from 0 to 10% by volume of 1-butene and from 0 to 50% by volume of 2-butenes, where the total amount is 100% by volume. Furthermore, small amounts of isobutane can be comprised.

The C₄ product gas stream can subsequently be separated by extractive distillation into a stream consisting essentially of n-butane and 2-butene and a stream consisting of butadiene. The stream consisting essentially of n-butane and 2-butene can be recirculated in its entirety or in part to the CA feed to the ODH reactor. Since the butene isomers of this recycled stream consist essentially of 2-butenes and these 2-butenes are generally oxidatively dehydrogenated to butadiene more slowly than is 1-butene, this recycled stream can go through a catalytic isomerization process before being fed into the ODH reactor. In this catalytic process, the isomer distribution can be brought to the isomer distribution present at thermodynamic equilibrium.

The extractive distillation can, for example, be carried out as described in “Erdöl und Kohle—Erdgas—Petrochemie”, volume 34 (8), pages 343 to 346, or “Ullmanns Enzyklopädie der Technischen Chemie”, volume 9, 4^{th} edition 1975, pages 1 to 18. For this purpose, the C₄ product gas stream is brought into contact with an extractant, preferably an N-methylpyrrolidone (NMP)/water mixture, in an extraction zone. The extraction zone is generally configured in the form of a scrubbing column comprising trays, random packing elements or ordered packings as internals. This generally has from 30 to 70 theoretical plates in order for a sufficiently good separation performance to be achieved. The scrubbing column preferably has a backwashing zone at the top of the column. This backwashing zone serves to recover the extractant comprised in the gas phase with the aid of a liquid hydrocarbon runback, for which purpose the overhead fraction is condensed beforehand. The mass ratio of extractant to C₄ product gas stream in the feed to the extraction zone is generally from 10:1 to 20:1. The extractive distillation is preferably operated at a temperature at the bottom in the range from 100 to 250° C., in particular at a temperature in the range from 110 to 210° C., a temperature at the top in the range from 10 to 100° C., in particular in the range from 20 to 70° C., and a pressure in the range from 1 to 15 bar, in particular in the range from 3 to 8 bar. The extractive distillation column preferably has from 5 to 70 theoretical plates.

Suitable extractants are butyrolactone, nitriles such as acetonitrile, propionitrile, methoxypropionitrile, ketones such as acetone, furfural, N-alkyl-substituted lower aliphatic acid amides such as dimethylformamide, diethylformamide, dimethylacetamide, diethylacetamide, N-formylmorpholine, N-alkyl-substituted cyclic acid amides (lactams) such as N-alkylpyrrolidones, in particular N-methylpyrrolidone (NMP). In general, alkyl-substituted lower aliphatic acid amides or N-alkyl-substituted cyclic acid amides are used. Dimethylformamide, acetonitrile, furfural and in particular NMP are particularly advantageous.

However, it is also possible to use mixtures of these extractants with one another, e.g. NMP and acetonitrile, mixtures of these extractants with cosolvents and/or tert-butyl ethers, e.g. methyl tert-butyl ether, ethyl tert-butyl ether, propyl tert-butyl ether, n-butyl or isobutyl tert-butyl ether. A particularly useful extractant is NMP, preferably in aqueous solution, preferably with from 0 to 20% by weight of water, particularly preferably with from 7 to 10% by weight of water, in particular with 8.3% by weight of water.

The overhead product stream from the extractive distillation column comprises essentially butane and butenes and small amounts of butadiene and is taken off in gaseous or liquid form. In general, the stream consisting essentially of n-butane and 2-butene comprises from 50 to 100% by volume of n-butane, from 0 to 50% by volume of 2-butene and from 0 to 3% by volume of further constituents such as isobutane, isobutene, propane, propene and Cs₅-hydrocarbons.

At the bottom of the extractive distillation column, a stream comprising the extractant, water, butadiene and small amounts of butenes and butane is obtained and this is fed to a distillation column. In this distillation column, butadiene is obtained at the top or as a side offtake stream. A stream comprising extractant and water is obtained at the bottom of the distillation column; the composition of the stream comprising extractant and water corresponds to the composition as is introduced into the extraction. The stream comprising extractant and water is preferably recirculated to the extractive distillation.

The extraction solution is transferred into a desorption zone in which the butadiene is desorbed from the extraction solution. The desorption zone can, for example, be in the form of a scrubbing column which has from 2 to 30, preferably from 5 to 20, theoretical plates and optionally a backwashing zone having, for example, 4 theoretical plates. This backwashing zone serves to recover the extractant comprised in the gas phase by means of a liquid hydrocarbon runback, for which purpose the overhead fraction is condensed beforehand. Ordered packings, trays or random packing elements are provided as internals. The distillation is preferably carried out at a temperature at the bottom in the range from 100 to 300° C., in particular in the range from 150 to 200° C., and a temperature at the top in the range from 0 to 70° C., in particular in the range from 10 to 50° C. The pressure in the distillation column is preferably in the range from 1 to 10 bar. In general, a lower pressure and/or higher temperature prevails in the desorption zone compared to the extraction zone.

The desired product stream obtained at the top of the column generally comprises from 90 to 100% by volume of butadiene, from 0 to 10% by volume of 2-butene and from 0 to 10% by volume of n-butane and isobutane. To purify the butadiene further, a further distillation as per the prior art can be carried out.

The invention is illustrated by the following examples.

### EXAMPLES

Catalyst Production

### Example 1

Two starting materials A and B are used.

Starting material A: 1 kg of milled chromium-free catalyst corresponding to example B (page 28) of DE 10 2007 004 961 A1. The catalyst has the stoichiometry Mo₁₂Co₇Fe₃Bi_{0.6}K_{0.08}Si_{1.6}Oₓ. The catalyst was milled by means of an opposed jet mill.

Starting material B: 14 g of Bayoxide C GN-M from Lanxess (Cr₂O₃)

The two starting materials are mixed at 190 rpm in an Amixon mixer for 15 minutes. The mixed powders are heated to 510° C. over a period of 3 hours 53 minutes using a linear ramp in a Nabertherm muffle furnace and then maintained at this temperature for 7 hours 47 minutes in a normal atmosphere using an inflow of 1000 standard l/h of air. After the calcination, an active composition having the calculated stoichiometry Mo₁₂Co₇Fe₃Bi_{0.6}K_{0.08}Cr_{0.6}Si_{1.6}Oₓ is obtained.

Support bodies (steatite rings having the dimensions 5×3×2 mm (external diameter×internal diameter×length) were coated with the precursor composition. Coating was carried out in a HiCoater LHC 25/36 (from Lödige, D-33102 Paderborn). This HiCoater was modified in order to allow continuous introduction of powder. This consisted of a funnel-shaped powder reservoir which was connected via a Tygon tube (internal diameter: 8 mm, external diameter 11.1 mm; from Saint-Gobain Performance, 89120 Charny, France) to the drum of the HiCoater. The drum radius was 18 cm. The depth of the drum was 20 cm. The axis about which the drum rotated was aligned horizontally. For the coating operation, 710 g of the catalytic active oxide composition powder were placed in the powder reservoir. Powder metering was carried out by continuous pressure metering. The pulse time valve was set to 30 ms and the pressure set was 0.6 bar above ambient pressure (˜1 atm). The powder in the funnel-shaped powder reservoir was continuously stirred during the coating operation in order to ensure uniform metering (running time of stirrer: 4 s, pause time of stirrer 1 s, modified V-shaped anchor stirrer, constructed in-house by BASF SE). The binder was an aqueous solution composed of 75% by weight of water and 25% by weight of glycerol. This was sprayed separately by means of a liquid metering device into the drum. The liquid was pumped by means of an HPLC pump from Watson-Marlow (model 323) into the metering arm located in the drum (spray pressure 3 bar, forming pressure 2 bar, mass flow: 1.8 g of glycerol/water solution (1:3)/min). The powder metering device and the liquid metering device were arranged parallel to one another. The nozzle from Schlick (DE) model 570/0 S75 installed on the metering arm and also the exit opening of the solids metering device which was likewise fastened underneath on the metering arm were aligned parallel at a spacing of 6 cm and by means of an angle measuring instrument at an angle of 40° to the horizontal. Powder metering was carried out outside the spray cone of the nozzle. The nozzle opening and exit opening of the solids metering device pointed in the direction of rotation of the drum. The drum rotated clockwise at 15 rpm during coating. Coating was carried out at 25° C. over a period of 30 minutes. The coated support materials were then dried at an air inlet temperature of 130° C. and an air outlet temperature of 81° C. for 27 minutes. They were then cooled in the static drum to 25° C. over a period of 30 minutes. During the coating operation, the powder fed in was mostly taken up on the surface of the support. The material which was not taken up was collected in a filter downstream of the drum. No formation of twins occurred and agglomeration of the finely divided oxidic composition was not observed.

The coated shaped support bodies were treated in a convection drying oven (from Memmert GmbH+Co. KG, model UM 400; internal volume=53 l; air flow=800 l/h) in order to remove the glycerol still present in the sample. For this purpose, the convection drying oven was heated to 300° C. (inclusive of the air temperature) over a period of 2 hours and then maintained at 300° C. for 2 hours. During drying, the material being dried was located in a layer (layer thickness=2 cm) on a perforated plate positioned centrally in the drying oven (the hole diameter of the through openings distributed uniformly over the perforated plate=0.5 cm; the opening ratio of the perforated plate was 60%; the total cross-sectional area of the perforated plate was 35 cm×26 cm=910 cm²). The convection drying oven was then cooled to 40-50° C. over a period of 2-3 hours and the sample was taken out. The hollow-cylindrical coated catalysts taken from the convection drying oven have, based on their total mass, an oxidic coating proportion of 16% by weight.

### Example 2 (Comparison)

A catalyst is produced as per example B (page 28) of DE 10 2007 004 961 A1. The catalyst has the stoichiometry Mo₁₂Co₇Fe₃Bi_{0.6}K_{0.08}Si_{1.6}Oₓ.

Support bodies (steatite rings having the dimensions 5×3×2 mm (external diameter×internal diameter×length) were coated with the precursor composition in a manner analogous to example 1.

The coated shaped support bodies were treated in a convection drying oven in a manner analogous to example 1. The hollow-cylindrical coated catalysts taken from the convection drying oven have, based on their total mass, an oxidic coating proportion of 15% by weight.

### Example 3 (Comparison)

The catalyst was produced as per the example on page 24 of WO 2014/08695, as follows:

2 solutions A and B were produced.

Solution A: 3200 g of water were placed in a 10 l stainless steel pot. While stirring by means of an anchor stirrer, 5.2 g of a KOH solution (32% by weight of KOH) were added to the initially charged water. The solution was heated to 60° C. 1066 g of an ammonium heptamolybdate solution ((NH₄)₆Mo₇O₂₄*4 H₂O, 54% by weight of Mo) were then added a little at a time over a period of 10 minutes. The resulting suspension was stirred for a further 10 minutes.

Solution B: 1771 g of a cobalt(II) nitrate solution (12.3% by weight of Co) were placed in a 5 l stainless steel pot and heated to 60° C. while stirring (anchor stirrer). 645 g of an iron(III) nitrate solution (13.7% by weight of Fe) were then added a little at a time over a period of 10 minutes while maintaining the temperature. The solution formed was stirred for a further 10 minutes. 619 g of a bismuth nitrate solution (10.7% by weight of Bi) were then added while maintaining the temperature. After stirring for a further 10 minutes, 109 g of chromium(III) nitrate were added in solid form a little at a time and the resulting dark red solution was stirred for a further 10 minutes.

While maintaining the 60° C., the solution B was pumped into the solution A by means of a peristaltic pump over a period of 15 minutes. During the addition and afterwards, the mixture was stirred by means of a high-speed mixer (Ultra-Turrax). After the addition was complete, the mixture was stirred for a further 5 minutes. 93.8 g of an SiO₂ suspension (Ludox; SiO₂ about 49%, from Grace) were then added and the mixture was stirred for a further 5 minutes.

The suspension obtained was spray dried in a spray dryer from NIRO (spray head No. FOA1, speed of rotation 25 000 rpm) over a period of 1.5 hours. The reservoir temperature was maintained at 60° C. during this time. The gas inlet temperature of the spray dryer was 300° C., and the gas outlet temperature was 110° C. The powder obtained had a particle size (do) of less than 40 μm.

The powder obtained was mixed with 1% by weight of graphite, compacted twice under a pressing pressure of 9 bar and broken up through a sieve having a mesh opening of 0.8 mm. The crushed material was once again mixed with 2% by weight of graphite and the mixture was pressed by means of a Kilian S100 tableting press to give rings having dimensions of 5×3×2 mm (external diameter×length×internal diameter).

The catalyst precursors obtained were calcined batchwise (500 g) in a convection oven from Heraeus, DE (model K, 750/2 S, internal volume 55 l). The following program was used for this purpose:
heat to 130° C. over 72 minutes, hold for 72 minutes
heat to 190° C. over 36 minutes, hold for 72 minutes
heat to 220° C. over 36 minutes, hold for 72 minutes
heat to 265° C. over 36 minutes, hold for 72 minutes
heat to 380° C. over 93 minutes, hold for 187 minutes
heat to 430° C. over 93 minutes, hold for 187 minutes
heat to 490° C. over 93 minutes, hold for 467 minutes

After the calcination, the catalyst having the calculated stoichiometry Mo₁₂Co₇Fe₃Bi_{0.6}K_{0.08}Cr_{0.5}Si_{1.6}Oₓ was obtained.

The calcined pellets were milled to a powder. Support bodies (steatite rings having the dimensions 5×3×2 mm (external diameter×internal diameter×length) were coated with the precursor composition in a manner analogous to example 1.

The coated shaped support bodies were treated in a convection drying oven in a manner analogous to example 1. The hollow-cylindrical coated catalysts taken from the convection drying oven had, based on their total mass, an oxidic coating proportion of 15% by weight.

### Example 4

Dehydrogenation Experiments

Dehydrogenation experiments were carried out in a screening reactor. The screening reactor was a salt bath reactor having a length of 120 cm and an internal diameter of 14.9 mm and a temperature sensor sheath having an external diameter of 3.17 mm. A multiple temperature sensor having seven measurement points was located in the temperature sensor sheath. The lowermost 4 measurement points had a spacing of 10 cm and the uppermost 4 measuring points had a spacing of 5 cm.

Raffinate II was metered in liquid form through a coriolis flow meter at about 10 bar and subsequently depressurized and vaporized in a heated vaporizer section. This gas was then mixed with nitrogen and fed into a preheater having a steatite bed. Water was introduced in liquid form and vaporized in a stream of air in a vaporizer coil. The air/water vapor mixture was combined with the N₂/raffinate 11 mixture in the lower region of the preheater. The completely mixed feed gas was then fed to the reactor, with an analysis stream for on-line GC measurement being able to be taken off. An analysis stream is likewise taken off from the product gas leaving the reactor and can be analyzed by on-line GC measurement or by means of an IR analyzer for the proportion by volume of CO and CO₂. A pressure regulating valve which set the pressure level in the reactor was installed downstream of the branch of the analysis line.

A 6.5 cm long after-bed consisting of 16 g of steatite balls having a diameter of from 3.5 to 4.5 mm was introduced on top of the catalyst grating at the lower end of the screening reactor. 120 g of the respective catalyst were then introduced into the reactor (67.5 cm bed height). The catalyst bed was followed by a 7 cm long pre-bed consisting of 16 g of steatite balls having a diameter of from 3 to 4 mm.

The catalysts were activated by being heated overnight at 400° C. in a mixture of oxygen, nitrogen and steam (10/80/10). The reactor was operated using from 100 to 250 standard l/h of a reaction gas having the composition 8% of butene, 2% of butane, 12% of oxygen, 5% of water, 73% of nitrogen at a salt bath temperature of 380° C. for 120 hours. The gas velocity was varied in order to alter the conversion. The salt bath temperature was 380° C. The product gases were analyzed by means of a GC. The conversion and selectivity data are shown in table 1.

The parameters conversion (X) and selectivity (S) calculated in the examples were determined as follows:

$$X = \frac{mol ( {butenes}_{i n} ) - mol ( {butenes}_{out} )}{mol ( {butene}_{m} )}$$

$$S = \frac{mol ( {butenes}_{out} ) - mol ( {butenes}_{i n} )}{mol ( {butenes}_{i n} ) - mol ( {butenes}_{out} )}$$

where mol(Xᵢₙ) is the molar amount of the component X at the reactor inlet, mol (Xₒᵤₜ) is the molar amount of the component X at the reactor outlet and butenes is the sum of 1-butene, cis-2-butene, trans-2-butene and isobutene.

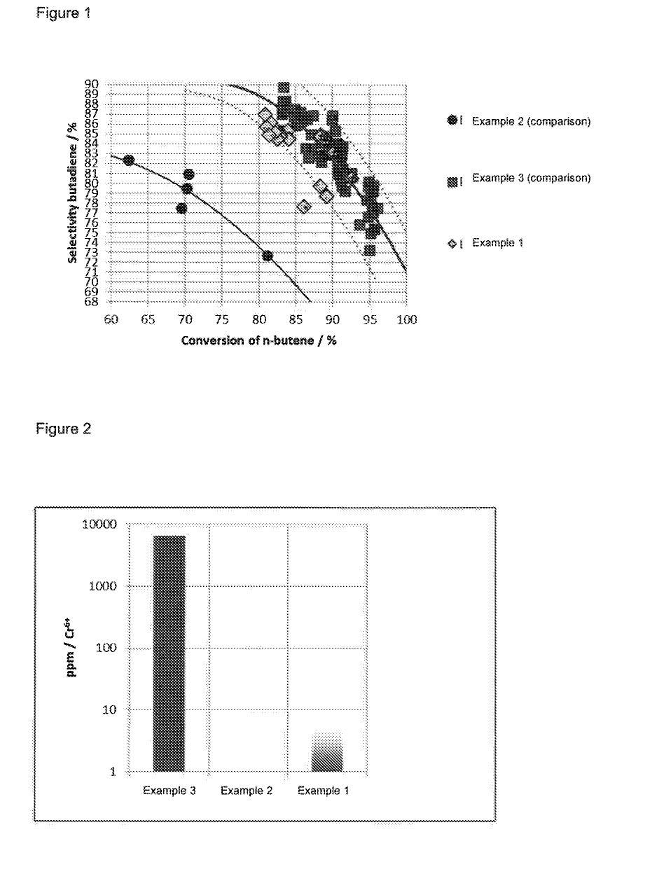

The conversion and selectivity data are shown in table 1 and reproduced in FIG. 1. The selectivities at about 85% conversion are compared. The Cr-comprising catalysts (examples 1 and 3) have, within the scatter of the measured values, the same selectivity for butadiene. The completely Cr-free catalyst of example 2 displays a significantly lower selectivity.

TABLE 1
Selectivities of the catalysts tested in examples 1 to 3

| Catalyst | Conversion (n-butene) | Selectivity (1,3-butadiene) |
|---|---|---|
| Example 1 | 85% | 84% |
| Example 2 | 85% | 70% |
| Example 3 | 85% | 86% |

FIG. 1 shows conversion-selectivity curves of the catalysts tested as per examples 1 to 3 (* denotes the value for the first operating stage after the start-up phase, # denotes the values of the first operating stage after the first regeneration phase).

### Example 5

Determination of Cr⁶⁺ in Catalyst Samples

Sample preparation: about 0.4 to 0.6 g of sample of the oxidic mixture are taken at an average temperature of 265° C. during the thermal treatment. In the case of example 1, sampling is carried out during the linear heating-up phase. In the case of examples 2 and 3, sampling is in each case carried out during the calcination in the course of the fourth heating-up phase in which the material is heated to 265° C. over 36 minutes and held at this temperature for 72 minutes. The samples taken in this way are weighed to within 0.1 mg into a 100 ml volumetric flask and made up to the mark with water. After stirring for 4 hours on a magnetic stirrer, the sample is filtered through a fluted filter (“water withdrawal”).

An aliquot of the solution is (depending on content) acidified with phosphoric acid and admixed with an excess over Cr⁶⁺ of 1,5-diphenylcarbazide (about 1 ml). Diphenylcarbazide forms a red-violet complex with dissolved Cr⁶⁺ and the concentration of this complex can be determined photometrically. The blank determination is carried out analogously, only without sample.

After allowing to stand for 15 minutes, the solutions are measured photometrically and the content is determined taking the blank into account.

FIG. 2 shows the proportions of hexavalent Cr(VI) formed as an intermediate at 265° C. for the catalysts of examples 1 to 3. In the case of example 1, the proportion of Cr(VI) compounds is below the detection limit of 10 ppm by weight. The catalyst of example 2 does not contain any chromium. In the case of example 3, the proportion of Cr(VI) compounds formed as intermediates is about 6200 ppm by weight (based on Cr(VI)).

1.-4. (canceled)

5. A process for producing a multimetal oxide catalyst comprising molybdenum, chromium and at least one further metal by mixing of a pulverulent multimetal oxide comprising molybdenum and the at least one further metal but no chromium with pulverulent chromium(III) oxide and thermally treating the resulting pulverulent mixture in the presence of oxygen at a temperature in the range from 350° C. to 650° C., wherein the multimetal oxide comprising molybdenum and the at least one further metal but no chromium has the general formula (I)
Mo₁₂BiₐFe_{b}Co_{c}Ni_{d}X¹_{f}X²_{g}Oₓ (I)
wherein:
X¹=W, Sn, Mn, La, Ce, Ge, Ti, Zr, Hf, Nb, P, Si, Sb, Al, Cd and/or Mg;
X²=Li, Na, K, Cs and/or Rb,
a=0.1 to 7;
b=0 to 10;
c=0 to 10;
d=0 to 10;
f=0 to 50;
g=0 to 2; and
x=a number determined by the valence and abundance of the elements other than oxygen in (I),
and the multimetal oxide catalyst comprising molybdenum, chromium and at least one further metal has the general formula (II)
Mo₁₂BiₐFe_{b}Co_{c}Ni_{d}CrₑX¹_{f}X²_{g}O_{y} (II)
where the variables have the following meanings:
X¹=W, Sn, Mn, La, Ce, Ge, Ti, Zr, Hf, Nb, P, Si, Sb, Al, Cd and/or Mg;
X²=Li, Na, K, Cs and/or Rb,
a=0.1 to 7;
b=0 to 10;
c=0 to 10;
d=0 to 10;
e=>0 to 5;
f=0 to 50;
g=0 to 2; and
y=a number determined by the valence and abundance of the elements other than oxygen in (II).

6. The process according to claim 5, wherein the production of the pulverulent multimetal oxide which comprises molybdenum and at least one further metal but no chromium comprises the steps (i) to (iv):
(i) producing a multimetal oxide precursor composition comprising molybdenum and at least one further metal but no chromium,
(ii) shaping of shaped bodies from the multimetal oxide precursor composition,
(iii) calcining the shaped bodies,
(iv) milling of the shaped bodies to give a pulverulent multimetal oxide.

7. The process according to claim 6, wherein the process comprises the steps (v) to (viii):
(v) mixing of the pulverulent multimetal oxide comprising molybdenum and at least one further metal but no chromium with pulverulent chromium(III) oxide,
(vi) thermally treating the pulverulent mixture in the presence of oxygen at a temperature of from 350 to 650° C. to give a pulverulent multimetal oxide catalyst comprising molybdenum, chromium and at least one further metal,
(vii) coating of a support body with the pulverulent multimetal oxide catalyst,
(viii) thermally treating the coated support body.

8. The process according to claim 5, wherein the multimetal oxide catalyst which comprises molybdenum, chromium and at least one further metal and has the general formula (IIa)
Mo₁₂BiₐFe_{b}Co_{c}Ni_{d}CrₑX¹_{f}X²_{g}O_{y} (IIa),
where the variables have the following meanings:
X¹=W, Sn, Mn, La, Ce, Ge, Ti, Zr, Hf, Nb, P, Si, Sb, Al, Cd and/or Mg;
X²=Li, Na, K, Cs and/or Rb,
a=0.1 to 7;
b=0 to 5;
c=0 to 10;
d=0 to 10;
e=0.01 to 5;
f=0 to 50;
g=0 to 2; and
y=a number determined by the valence and abundance of the elements other than oxygen in (II).

9. The process according to claim 5, wherein in formula (I):
a=0.3 to 1.5;
b=2 to 4;
c=3 to 10;
d=0;
f=0.1 to 10; and
g=0.01 to 1; and wherein in formula (II):
a=0.3 to 1.5;
b=2 to 4;
c=3 to 10;
d=0;
e=0.1 to 2;
f=0.1 to 10; and
g=0.01 to 1.
